(19) United States
(12) Patent Application Publication
(10) Pub. No.: US 2024/0245427 A1
(43) Pub. Date: Jul. 25, 2024

(54) CIRCUMCISION EQUIPMENT

(71) Applicant: Wuhu ShangRing Technology Co., Ltd, Wuhu (CN)

(72) Inventors: Shujie Xia, Shanghai (CN); Huarong Yu, Beijing (CN); Jingjing Shang, Wuhu (CN); Jianzhong Shang, Wuhu (CN)

(21) Appl. No.: 18/624,119

(22) Filed: Apr. 2, 2024

Related U.S. Application Data
(62) Division of application No. 16/531,139, filed on Aug. 5, 2019, now Pat. No. 11,980,390.
Application No. 16/531,139 is a continuation of application No. PCT/CN2018/074988, filed on Feb. 1, 2018.

(30) Foreign Application Priority Data
Feb. 4, 2017 (CN) 201710063339.X
Feb. 4, 2017 (CN) 201710063340.2
Feb. 4, 2017 (CN) 201710065136.4
Feb. 4, 2017 (CN) 201710065137.9
Feb. 4, 2017 (CN) 201710065138.3
Feb. 4, 2017 (CN) 201710065139.8
Feb. 4, 2017 (CN) 201710065140.0
Feb. 4, 2017 (CN) 201710065146.8
Feb. 4, 2017 (CN) 201710065147.2
Feb. 4, 2017 (CN) 201710065148.7
Feb. 4, 2017 (CN) 201710065149.1
Feb. 4, 2017 (CN) 201710065150.4
Feb. 4, 2017 (CN) 201720109034.3
Apr. 18, 2017 (CN) 201710250780.9
Apr. 18, 2017 (CN) 201710250951.8
Apr. 19, 2017 (CN) 201710254989.2
Apr. 19, 2017 (CN) 201710255021.1
Apr. 19, 2017 (CN) 201710255023.0
Apr. 19, 2017 (CN) 201710255025.X
Apr. 24, 2017 (CN) 201710273894.5
Apr. 24, 2017 (CN) 201710273895.X
Apr. 24, 2017 (CN) 201710274306.X
Apr. 24, 2017 (CN) 201710274307.4
Apr. 24, 2017 (CN) 201710274308.9
Apr. 24, 2017 (CN) 201710274309.3
Apr. 24, 2017 (CN) 201710274396.2
Apr. 24, 2017 (CN) 201710274397.7
Apr. 24, 2017 (CN) 201710274398.1
Apr. 24, 2017 (CN) 201710274399.6
Apr. 24, 2017 (CN) 201710274400.5
Apr. 24, 2017 (CN) 201710274426.X
Aug. 18, 2017 (CN) 201710709597.0

(51) Int. Cl. A61B 17/326; A61B 17/068; A61B 17/115

(52) U.S. Cl. CPC A61B 17/326; A61B 17/0682; A61B 17/1155

(57) ABSTRACT

The present invention relates to surgical devices, particularly to a circumcision apparatus, enable the circumcision apparatus to be suitable for different surgery objects and environments.

### CROSS REFERENCE TO RELATED APPLICATIONS

The present application is a Divisional Application of U.S. patent application Ser. No. 16/531,139 filed on Aug. 5, 2019, which is a Continuation Application of PCT Application No. PCT/CN2018/074988 filed on Feb. 1, 2018, which claims the benefit of Chinese patents application Ser. Nos. 20/171,0063339.X, 201710065146.8, 201710065140.0, 201710065139.8, 201710065138.3, 201710065150.4, 201710065149.1, 201710065148.7, 201710065147.2, 201710063340.2, 201710065137.9, 201710065136.4, 201720109034.3 filed on Feb. 4, 2017, Chinese patents application Ser. Nos. 20/171,0250780.9 and 201710250951.8 filed on Apr. 18, 2017, Chinese patents application Ser. Nos. 20/171,0254989.2, 201710255021.1, 201710255023.0 and 201710255025.X filed on Apr. 19, 2017, Chinese Patent Application Nos. 201710274426.X, 201710274400.5, 201710274399.6, 201710274398.1, 201710274397.7, 201710274396.2, 201710274309.3, 201710274308.9, 201710274307.4, 201710274306.X, 201710273895.X and 201710273894.5 filed on Apr. 24, 2017, and Chinese patent application Ser. No. 20/171,0709597.0 filed on Aug. 18, 2017. All the above are hereby incorporated by reference.

### FIELD OF THE INVENTION

The present invention relates to surgical devices, particularly to a circumcision apparatus and more particularly to an advancing and self-locking device and a circumcision device, as well as an assembling method and an application method of the advancing and self-locking device.

### BACKGROUND OF THE INVENTION

Refer to the advancing and self-locking device and a circumcision device, as well as the assembling method and the application method. The redundant prepuce or phimosis is one of the causes of male urinary system infections and sexually transmitted diseases. The redundant prepuce or phimosis can cause urinary tract infections, resulting in chronic prostatitis, with a series of symptoms, such as pains in back and waist, impotence and premature ejaculation. Therefore, the removal of the redundant prepuce is a good way to prevent these diseases.

Traditionally, surgical removal of the phimosis or redundant prepuce has the main technical points of removal of excess prepuce, hemostasis, and apposition suture of cut edges to skin. A postoperative patient cannot move around, suffers unbearable pain in each change of ointment and endures huge pain when stitches are taken out finally. In addition, incomplete ligating hemostasis will cause prepuce hematoma, thus requiring a surgical treatment again. Furthermore, since the prepuce removal and the hemostasis process are performed separately, the operation time is prolonged, and the patient's panic is exacerbated.

A therapeutic method of applying a laser and high-frequency electric surgical knife technology to circumcision has been developed at present. Although this therapeutic method substitutes for scissors cutting and makes a bleeding spot coagulated, the patient's tissues will be burnt and susceptible to infection.

Then, a circumcision device in which a prepuce incision is sutured using a suturing nail at one time was developed in this field. However, the circumcision device in the prior art seldom uses a special advancing and self-locking device, and therefore, the states (an initial assembly state, an assembly completion state, an initial use state and a use completion state) of the circumcision device cannot be accurately judged, and it is inconvenient to distinguish the used circumcision device from the unused. Accordingly, there is the risk of cross infection caused by repeated use among patients, easily resulting in misoperations and medical accidents.

### SUMMARY OF THE INVENTION

Refer to the advancing and self-locking device and a circumcision device, as well as the assembling method and the application method.

An objective of the present invention is to provide an advancing and self-locking device and a circumcision device, as well as an assembling method and an application method of the advancing and self-locking device. With respect to the problems in the prior art, the present invention can conveniently distinguish the used circumcision device from the unused ones, and accordingly, the cross infection caused by repeated use among patients is avoided to reduce the occurrence of medical accidents. The self-locking device provided by the present invention uses a buckle structure and a convex plate structure, and is simple in structure, clear in indications, reliable in locking, and easy to operate when being applied to the circumcision device. The specific technical solution is as follows.

An advancing and self-locking device, comprising a hook and a convex plate, wherein the convex plate is arranged on the inner wall of a sheathed handle; the hook is arranged on a sheathed core; the sheathed core is arranged in the sheathed handle in a penetrating manner; the hook and the convex plate are matched with each other so as to allow the sheathed core to only move in the sheathed handle axially and unidirectionally.

Further, a stage of convex plate is arranged on the inner wall of the sheathed handle in a movement direction of the sheathed core, thereby forming a locking position.

Further, a plurality of stages of convex plates is arranged on the inner wall of the sheathed handle in a movement direction of the sheathed core, thereby forming a plurality of locking positions.

Further, a first-stage convex plate and a second-stage convex plate are sequentially arranged on the inner wall of the sheathed handle in a movement direction of the sheathed core; a first locking position is formed at one side of the first-stage convex plate, which is close to the second-stage convex plate; a second locking position is formed at one side of the second-stage convex plate, which is away from the first-stage convex plate.

Further, a chamfer is arranged at one side of the first-stage convex plate, which is away from the second-stage convex plate.

Further, a sleeve is arranged in a region, which corresponds to a position where the convex plate is located, on the outer wall of the sheathed handle.

Further, the sleeve is rotatably arranged on the outer wall of the sheathed handle, thereby allowing or preventing the sheathed core to move or from moving in the sheathed handle axially and unidirectionally.

A circumcision device uses the advancing and self-locking device described above.

Further, the circumcision device further comprises a main device, a driving device, a removing device and a positioning device, wherein the driving device is mounted on the main device and connected to the removing device and serves to drive the removing device; the removing device is mounted on the main device, is close to or connected to the positioning device and can serve to remove and/or anastomose the prepuce positioned on the positioning device; the positioning device is mounted on the main device and serves to position glans penis and/or prepuce.

The main device comprises the sheathed handle, the sheathed core, a knob and a screw rod, wherein the sheathed core is fully or partially arranged in the sheathed handle in a penetrating manner; the knob is arranged at the tail end of the sheathed core; one end of the screw rod is connected to the knob and arranged in the sheathed core in a penetrating manner.

The driving device comprises handles, connecting rods, pins and a spring, wherein the front ends of the handles are connected to the sheathed handle respectively; the connecting rods are connected to the middle sections of the handles respectively; the connecting rods are connected to the sheathed core respectively through the pins.

The positioning device comprises a lower sleeve, an upper sleeve, an inner sleeve, an inner sleeve washer and an upper sleeve washer, wherein the lower sleeve and the upper sleeve form an outer sleeve together; the inner sleeve is arranged in the outer sleeve; the inner sleeve is provided with the inner sleeve washer; the upper sleeve is correspondingly provided with the upper sleeve washer. The removing device comprises a blade and a thimble which are arranged between the outer sleeve and the inner sleeve.

An assembling method and an application method for the advancing and self-locking device of the circumcision device comprise the following steps:
(1) rotating the sleeve on the sheathed handle, thereby allowing the sheathed core to move;
(2) advancing the sheathed core axially in the sheathed handle, till the hook moves across the first-stage convex plate with the chamfer;
(3) stopping the hook by the second-stage convex plate after moving across the first-stage convex plate; loosening the sheathed core at this moment, such that the sheathed core moves backwards under the action of a spring, till the hook is blocked by the first-stage convex plate, thereby positioning the hooking the first locking position to accomplish the assembly of the self-locking device;
(4) mounting the handles and the connecting rods on a lock handle and a lock core;
(5) during a surgery, pressing, by an operator, the handles to drive the sheathed core to further advance axially in the sheathed handle, till the hook moves across the second-stage convex plate, thereby accomplishing the circumcision of the prepuce; and
(6) at the end of the surgery, loosening the handles, such that the sheathed core moves backwards under the action of the spring, till the hook is blocked by the second-stage convex plate, thereby positioning the hook in the second locking position to accomplish the self-locking of the circumcision device.

The present invention makes improvements in the following aspects and achieves remarkable effects.
(1) The self-locking structure in which the hooks and the two stages of convex plates are matched is designed, such that state judgment and position locking of the circumcision device during assembling and use can be effectively realized.
(2) The chamfer design is adopted at the outer surface of the first-stage convex plate, such that the resistance can be effectively reduced, and the circumcision device can be manually assembled conveniently.
(3) By designing the resistance of the second-stage convex plate to be larger than that of the first-stage convex plate, followed by the elasticity of the spring, the hook can be enabled to move across the second-stage convex plate to accomplish the circumcision of the prepuce only with the help of the lever effect of the handles, thereby avoiding the misoperations occurring in the manual assembly process, accurately positioning the hook in the first locking position and accurately accomplishing the assembly of the self-locking device.
(4) After the surgery, the hook is positioned in the second locking position, and the handles cannot reset to an initial state, thereby accomplishing the self-locking of the circumcision device. The user can judge whether an apparatus has been used accurately by observing the positions of the handles, which is simple, intuitive and high in accuracy.
(5) By means of the structural configuration of the present invention, relatively lower resistance is provided at the first-stage convex plate, and a relatively higher resistance is provided at the second-stage convex plate, such that the start and end of assembly can be determined conveniently during assembly.

### BRIEF DESCRIPTION OF THE DRAWINGS

Refer to the advancing and self-locking device and a circumcision device, as well as the assembling method and the application method.

FIG. 1 is an overall schematic diagram of a circumcision device provided with an advancing and self-locking device of the present invention;

FIG. 2 is a sectional view of the circumcision device provided with the advancing and self-locking device of the present invention;

FIG. 3 is an enlarged schematic diagram of a self-locking device in FIG. 2;

FIG. 4 is an enlarged schematic diagram of a part B in FIG. 2;

FIG. 5 is an exploded schematic diagram of the circumcision device provided with the advancing and self-locking device of the present invention; in the drawings, the reference signs represent the following components: sheathed handle 1; identification point 1-1; sheathed core 2; sleeve 3; hook 4; first-stage convex plate 5; second-stage convex plate 6; handle 10; knob 11; connecting rod 12; pin 13; screw rod 14; spring 15; lower sleeve 16; upper sleeve 17; inner sleeve 18; inner sleeve washer 19; upper sleeve washer 20; blade 21; thimble 22.

### SPECIFIC DESCRIPTION OF THE EMBODIMENTS

Refer to the advancing and self-locking device and a circumcision device, as well as the assembling method and the application method.

The present invention is described in detail below on the basis of the drawings, which is a preferred embodiment of the present invention.

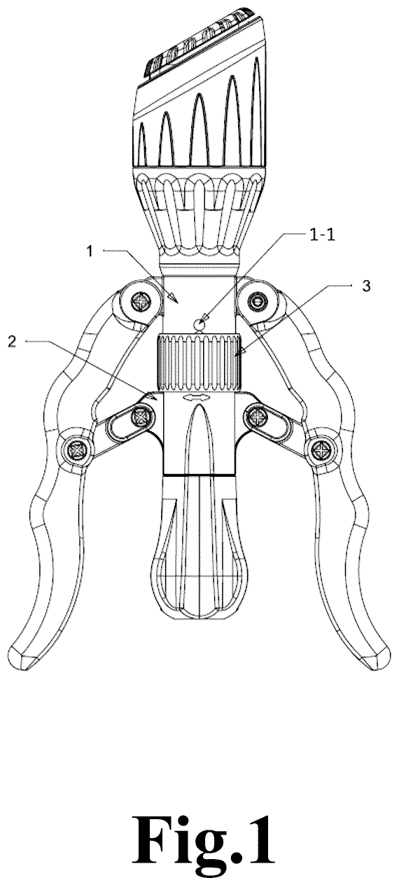

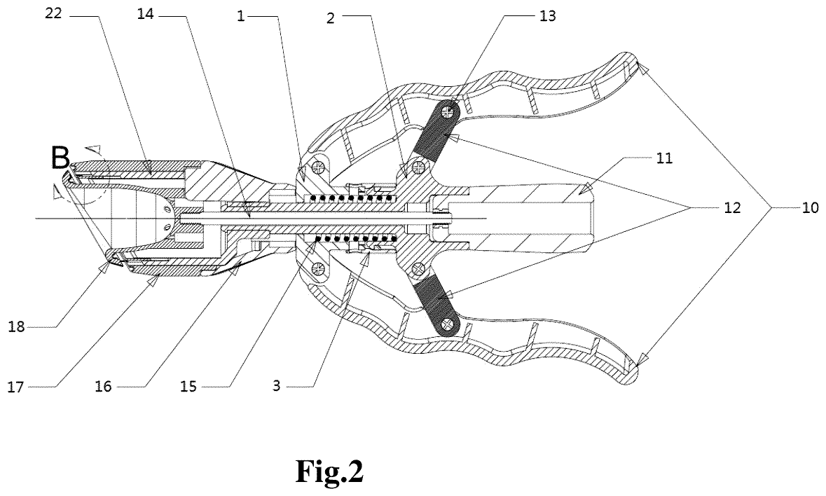

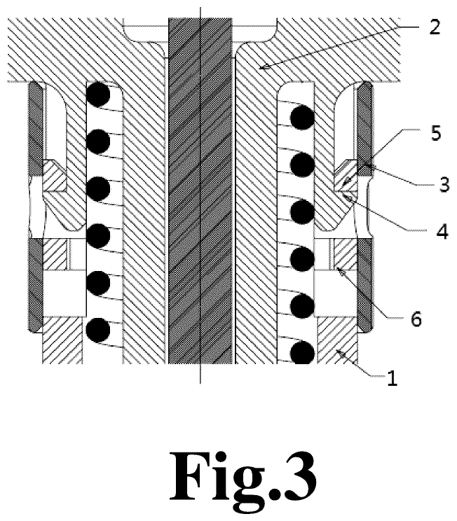

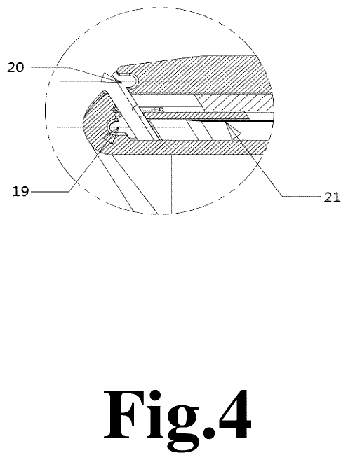

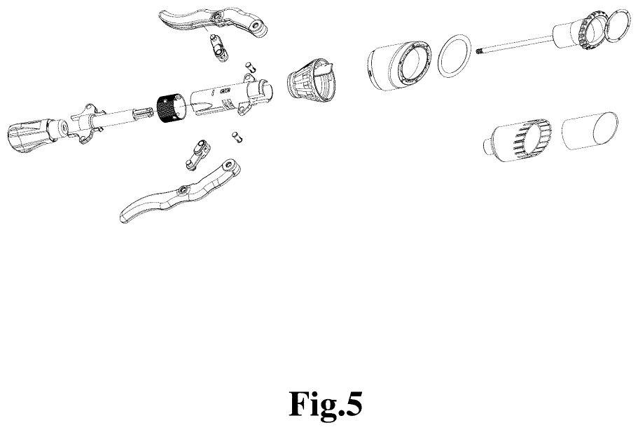

Referring to FIGS. 1-5, in the preferred embodiment, the sheathed handle 1 is provided with an identification point 1-1 and is sleeved with a sleeve 3.

During assembly, the sleeve 3 is rotated, such that a mark on the sleeve 3 leaves from the identification point 1-1, and the sheathed core 2 at this moment can be pushed to move. Then, the sheathed core 2 is pushed to advance axially in the sheathed handle 1, till the hook 4 moves across the first-stage convex plate 5. When an assembler continues to push the hook 4 to prop against the second-stage convex plate 6, but not moving across the same, the sheathed core 2 is loosened, such that the sheathed core 2 is positioned at the inner side, i.e., a first locking position, of the first-stage convex plate 5 under the action of a spring 15.

Handles 10 and connecting rods 12 are then mounted on the sheathed core 2 and the sheathed handle 3 to accomplish the assembly.

During use, the user presses the handles 10 to drive the sheathed core 2 to advance axially in the sheathed handle 3, till the hook 4 moves across the second-stage convex plate 6 to accomplish the circumcision of the prepuce.

At the end of a surgery, the user loosens the handles 10, the sheathed core 2 is positioned at the inner side of the second-stage convex plate 6 under the action of the spring 15, i.e., the hook 4 is positioned in the second locking position, and the handles 10 at the moment cannot reset to an initial state, thereby accomplishing the self-locking of the circumcision device. The user can accurately judge whether an apparatus has been used by observing the positions of the handles 10, which is simple, intuitive, high in accuracy and effectively avoids medical accidents.

Exemplary descriptions are given for the present invention with reference to the drawings in the above. Obviously, the present invention is not limited to the above implementation manners. Any improvement using the methods, concepts and technical solutions of the present invention or direct application of the present invention to other occasions without modifications shall fall into the protection scope of the present invention.

1. An advancing and self-locking device, comprising a hook (4) and a convex plate, wherein the convex plate is arranged on the inner wall of a sheathed handle (1); the hook (4) is arranged on a sheathed core (2); the sheathed core (2) is arranged in the sheathed handle (1) in a penetrating manner; the hook (4) and the convex plate are matched with each other so as to allow the sheathed core (2) to only move in the sheathed handle (1) axially and unidirectionally.

2. The advancing and self-locking device according to claim 1, wherein
a stage of convex plate is arranged on the inner wall of the sheathed handle (1) in a movement direction of the sheathed core (2), thereby forming a locking position; or,
a plurality of stages of convex plates is arranged on the inner wall of the sheathed handle (1) in a movement direction of the sheathed core (2), thereby forming a plurality of locking positions.

3. The advancing and self-locking device according to claim 2, wherein a first-stage convex plate (5) and a second-stage convex plate (6) are sequentially arranged on the inner wall of the sheathed handle (1) in the movement direction of the sheathed core (2); a first locking position is formed at one side of the first-stage convex plate (5), which is close to the second-stage convex plate (6); a second locking position is formed at one side of the second-stage convex plate (6), which is away from the first-stage convex plate (5).

4. The advancing and self-locking device according to claim 3, a chamfer is arranged at one side of the first-stage convex plate (5), which is away from the second-stage convex plate (6).

5. The advancing and self-locking device according to claim 4, wherein a sleeve (3) is arranged in a region, which corresponds to a position where the convex plate is located, on the outer wall of the sheathed handle (1); preferably, the sleeve (3) is rotatably arranged on the outer wall of the sheathed handle (1), thereby allowing or preventing the sheathed core (2) to move or from moving in the sheathed handle (1) axially and unidirectionally.

6. A circumcision device adopting the advancing and self-locking device according to claim 1.

7. The circumcision device according to claim 6, wherein
a stage of convex plate is arranged on the inner wall of the sheathed handle (1) in a movement direction of the sheathed core (2), thereby forming a locking position; or,
a plurality of stages of convex plates is arranged on the inner wall of the sheathed handle (1) in a movement direction of the sheathed core (2), thereby forming a plurality of locking positions.

8. The circumcision device according to claim 7, wherein a first-stage convex plate (5) and a second-stage convex plate (6) are sequentially arranged on the inner wall of the sheathed handle (1) in the movement direction of the sheathed core (2); a first locking position is formed at one side of the first-stage convex plate (5), which is close to the second-stage convex plate (6); a second locking position is formed at one side of the second-stage convex plate (6), which is away from the first-stage convex plate (5).

9. The circumcision device according to claim 8, a chamfer is arranged at one side of the first-stage convex plate (5), which is away from the second-stage convex plate (6).

10. The circumcision device according to claim 9, wherein a sleeve (3) is arranged in a region, which corresponds to a position where the convex plate is located, on the outer wall of the sheathed handle (1); preferably, the sleeve (3) is rotatably arranged on the outer wall of the sheathed handle (1), thereby allowing or preventing the sheathed core (2) to move or from moving in the sheathed handle (1) axially and unidirectionally.

11. The circumcision device according to claim 10, further comprising a main device, a driving device, a removing device and a positioning device, wherein
the driving device is mounted on the main device and connected to the removing device and serves to drive the removing device;
the removing device is mounted on the main device, is close to or connected to the positioning device and can serve to remove and/or anastomose the prepuce positioned on the positioning device;
the positioning device is mounted on the main device and serves to position glans penis and/or prepuce.

12. The circumcision device according to claim 11, wherein the driving device comprises handles (10), connecting rods (12), pins (13) and a spring (15), wherein the front ends of the handles (10) are connected to the sheathed handle (1) respectively; the connecting rods (12) are connected to the middle sections of the handles (10) respectively; the connecting rods (12) are connected to the sheathed core (2) respectively through the pins (13).

13. An assembling method for the advancing and self-locking device of the circumcision device according to claim 12, comprising the following steps:
(1) rotating the sleeve (3) on the sheathed handle (1), thereby allowing the sheathed core (2) to move;
(2) advancing the sheathed core (2) axially in the sheathed handle (1), till the hook (4) moves across the first-stage convex plate (5) with the chamfer;
(3) stopping the hook (4)by the second-stage convex plate (6) after moving across the first-stage convex plate (5); loosening the sheathed core (2) at this moment, such that the sheathed core (2) moves backwards under the action of the spring (15), till the hook (4) is blocked by the first-stage convex plate (5), thereby positioning the hook (4) in the first locking position to accomplish the assembly of the self-locking device; and
(4) mounting the handles (10) and the connecting rods (12) on a lock handle and a lock core.

14. An application method for the advancing and self-locking device of the circumcision device according to claim 12, comprising the following steps:
(1) during a surgery, pressing, by an operator, the handles (10) to drive the sheathed core (2) to further advance axially in the sheathed handle (1), till the hook (4) moves across the second-stage convex plate (6), thereby accomplishing the circumcision of the prepuce; and
(2) at the end of the surgery, loosening the handles (10), such that the sheathed core (2) moves backwards under the action of the spring (15), till the hook (4) is blocked by the second-stage convex plate (6), thereby positioning the hook (4) in the second locking position to accomplish the self-locking of the circumcision device.
